(19) United States
(12) Patent Application Publication
(10) Pub. No.: US 2025/0101405 A1
(43) Pub. Date: Mar. 27, 2025

(54) METHODS FOR PRODUCTION OF MICROALGINATE BEADS AND USES FOR ENCAPSULATION

(71) Applicant: Battelle Memorial Institute, Columbus, OH (US)

(72) Inventors: Timothy Lane, Columbus, OH (US); Veronica Fulwider, Columbus, OH (US)

(21) Appl. No.: 18/898,548

(22) Filed: Sep. 26, 2024

Related U.S. Application Data
(60) Provisional application No. 63/585,598, filed on Sep. 26, 2023.

(51) Int. Cl. C12N 11/04; C12N 11/10

(52) U.S. Cl. CPC C12N 11/04; C12N 11/10

(57) ABSTRACT

The invention relates to scale-up of alginate microbeads encapsulating biological actives comprising proteins and microorganisms. The invention provides a system and method for reliably producing micrometer size range alginate beads, in large volumes, to advance and sustain agriculture. Using any desired microorganism, the system and method provides a successful encapsulation approach, retaining the viability of the microorganism.

### RELATED APPLICATIONS

This application claims the priority benefit of U.S. Provisional Patent Application Ser. No. 63/585,598 filed 26 Sep. 2023.

### FIELD OF THE INVENTION

The present invention is directed to the system and method for large-scale production of microalginate beads and use for encapsulation of proteins and microorganisms.

### INTRODUCTION

There is an increased interest in the use of beneficial microorganisms or proteins in agriculture, horticulture, forestry and environmental management to improve plant growth and conserve agriculture. A principal drawback to this approach is the challenge of producing large quantities of beneficial microorganisms that can retain viability to positively affect plant health. Therefore, there is a critical need for a method that is successful when scaled-up, in preserving viability and longevity of biological actives, and for deployment of the technology for practical applications.

Alginate bead encapsulation has been traditionally used to stabilize and deliver a variety of active ingredients. An established method for producing alginate beads involves dripping a solution of sodium alginate (with active ingredients dissolved or dispersed within) through a nozzle, or nozzle array, into an aqueous bath containing a dissolved salt of a multivalent cation such as calcium, aluminum or iron. When droplets of alginate solution penetrate the surface of the “curing bath” a rapid ion exchange occurs, instantly crosslinking the alginate. This method produces beads having diameters larger than 1.0 mm.

We have shown (US 2018/0142229 A1) that encapsulation of biological agents using a biocompatible polymer, stabilizes the payload, and achieves bead sizes in the millimeter size range. Smaller diameter beads in the micrometer size range have been achieved, with agrochemicals as the payload (US 2018/0289001). Additionally, using alginate for encapsulation, we have successfully encapsulated biological actives, producing beads sized in the micrometer range (US 2021/0259255 A1). However large-scale production of these beads has not been investigated.

Lab scale methods (and some low-volume, high-value production processes) utilize one-drop-at-a-time techniques involving nozzles that deliver single-file streams of droplets that are atomized using ultrasonic vibration and/or imparting an electrostatic charge to the liquid streams. In any case, the throughput per nozzle is exceedingly small and scale-up to substantial volumes requires inordinate number of nozzles.

To address the challenges that hinder agricultural benefit and address problems linked to the continuous development of new and successful methods, an object of the present invention is therefore to provide a method and system for large-scale encapsulation of biological actives, as alginate microbeads, in biologically compatible size ranges from 50 μm-100 μm.

### SUMMARY OF THE INVENTION

The present disclosure is generally directed to a system and method for large-scale generation of encapsulated microorganisms and proteins.

The invention provides a system or method for generation of alginate microbeads encapsulating biological actives, comprising passing a solution comprising a biological active and an amphiphilic compound onto a spinning disk to generate microbeads and capturing the microbeads in a curing bath. The “biological actives” comprise microrganisms or compounds that create a physiological effect on a plant, animal or other microorganism. The term “amphiphilic” has the conventional meaning having both hydrophilic and lipophilic properties.

The invention may be further characterized by one or any combination of the following: wherein the “biological active” comprises a protein; wherein the biological active is selected from the group comprising: Bradyrhizobium japonicum, Pseudomonas protegens, Rhizobium tropici, Rhizobium leguminosarum, Ensifer meliloti, Burkholderia phytofirmans, Azospirillum brasilense, Rhodopseudomonas acidophila, Rhodotorula sp., Penicillium bilaiae, and combinations thereof; wherein the amphiphilic compound is a polysaccharide; wherein the amphiphilic compound is selected from the group consisting of sodium alginate, potassium alginate, barium alginate, calcium alginate, magnesium alginate, strontium alginate, and a combination thereof; wherein the curing bath comprises an aqueous solution comprising calcium chloride; wherein the curing bath comprises an aqueous solution comprising calcium acetate; wherein at least 95 mass % of the microbeads are in the size range of 1 to 800 μm, or 1 to 500 μm; wherein at least 30 mass %, or at least 50 mass %, at least 70 mass %, or at least 90 mass % of the microbeads are in the size range of 50 to 100 μm; wherein at least 80 mass %, or at least 90 mass % of the microbeads are spherical; wherein the spinning disk comprises a serrated cup; wherein the spinning disk retains the viability of the microorganisms; wherein at least 30 mass %, or at least 50 mass %, at least 70 mass %, or at least 90 mass % of the microbeads exhibit at least 95% viability; comprising generating 1000 liters of encapsulated biological actives while retaining at least 80% of activity.

Various embodiments contemplated herein may include, but need not be limited to, one or more of the following:

Embodiment 1: Large-scale generation of alginate microbeads is achieved by the passage of sodium alginate solution and biological actives through a spinning disc sprayer and contact of the sprayed droplets with a curing bath.

Embodiment 2: The biological actives in embodiment 1, wherein the biological actives include microorganisms and/or proteins that are combined with the alginate solution in any order, prior to passage through the spinning disc sprayer.

Embodiment 3: The microorganisms are cultured by fermentation, under conditions that support growth and viability.

Embodiment 4: The curing bath comprises calcium chloride solution and is set-up with or without agitation.

Embodiment 5: Generation of alginate microbeads encapsulating biological actives, wherein the alginate microbeads are created upon contact of the mixture with the curing bath, crosslinking alginate to build the microbead shell.

The invention can provide a method to produce functional alginate microbeads on a large-scale, having a diameter of 100 μm or less. Preferably, the curing bath comprises a calcium chloride solution that is set-up with or without agitation. It was surprisingly found that a rotary atomizer consistently produces droplets in the desired micrometer size range at a much higher flow rate, which is required for a functional scale-up process.

In a further aspect, the method operates at scale to produce more than 1000 gal (3800 L) of encapsulated formulations without loss of biological activity.

This invention can be used to reliably scale-up production of alginate microbeads for encapsulation and delivery of biologicals.

The invention includes any of the apparatus, methods, systems (apparatus plus fluids and, optionally conditions, or data described herein. The method, system, or apparatus may be further characterized by ±10% or ±20% or ±30% of any of the properties and/or measurements described herein. The invention is further elucidated in the examples below. In some preferred embodiments, the invention may be further characterized by any selected descriptions from the examples, for example, within ±30%, ±20% (or within ±10%) of any of the values in any of the examples, tables or figures. “All ranges are inclusive and combinable. For example, when a range of “1 to 5’ is recited, the recited range should be construed as including ranges “1 to 4”, “1 to 3”, “1-2”, “1-2 & 4-5”, “1-3 & 5”, “2-5”, any of 1, 2, 3, 4, or 5 individually, and the like.”

As is standard patent terminology, the term “comprising” means “including” and does not exclude additional components. Any of the inventive aspects described in conjunction with the term “comprising” also include narrower embodiments in which the term “comprising” is replaced by the narrower terms “consisting essentially of” or “consisting of.”

### Glossary

In the examples, the term “alginate solution” or “alginate” refers to a 2 wt. % solution of sodium alginate. Generally, an alginate solution preferably comprises 0.5 to 5 wt % sodium alginate.

The term “culturing” refers to growing a population of cells, e.g., microbial cells, under suitable conditions for growth, in a liquid or solid medium.

In the examples, the term “curing bath” as used herein refers to a 200 mM solution of calcium chloride, or in some cases calcium acetate (to mitigate the corrosive effects of Cl⁻).

As used herein, the term “growth” or “microbial growth” means any measurable change attributable to/or occurring within the life history of an organism. The measurable change can refer to an increase in attributes such as mass, cell divisions (e.g., binary fission events or cell doubling resulting in the production of daughter cells), cell number, cell metabolism products, or any other experimentally observable attribute of a microorganism.

The term “recipe”, as used herein may include any traditional microbiological culture medium that may be known to a person of ordinary skill in the art and can further include any growth (or selective) medium comprising any combination of medium components, whether defined or undefined (complex). Examples of medium components and classes of components include carbon sources, nitrogen sources, amino acids, extracts, salts, metal ions, cofactors, vitamins, dissolved gasses, and the like. Similarly, a “recipe” can include various components that might be added to a medium to influence the growth of a microorganism, such as selective and non-selective antimicrobial agents, modulating agents (i.e., agents that may alter microorganism growth, or enrichment agents (e.g., substance that may be required for auxotrophic microorganisms, such as hemin, or substances that may be required by fastidious organisms) or other components that may encourage microorganism growth.

The term “growth conditions” of the microbe refers to one or more conditions suitable for growth. For example, a “condition” can include one or more parameters required for/or beneficial to microorganism growth. A “condition” can also include other environmental parameters separate from the composition of a culture medium, such as light, pressure, temperature, aerobic/anaerobic and the like. Similarly, a condition can include any of a variety of other parameters that might occur or be imposed, such as: a host organism defensive material or cell (e.g., human defensing proteins, complement, antibody, macrophage cell, etc.), a surface adherent material (i.e., surfaces intended to permit growth, etc.), a physiological, metabolic, or gene expression modulating agent, a physiological salt, metabolite, or metabolic waste material (such as may be produced by living microorganisms or used to simulate late-stage culture growth conditions (i.e., stationary phase conditions), a reduction in nutrient media (simulating, for example, stationary phase conditions). Furthermore, a “condition” may be static (e.g. a fixed concentration or temperature) or dynamic (e.g. time-varying, to simulate pharmacokinetic behavior of intermittent infusions; or to simulate any endogenous or exogenous process affecting microbe response). These definitions of “condition” are intended to be illustrative, rather than exhaustive, and, as used herein, a “condition” can include any endogenous or exogenous parameter that may influence a microorganism.

As used herein, the term “microorganism”, used interchangeably with “microbial cell”, refers to one of the following classes: bacteria, fungi, algae, eukaryote, archaea, protozoa and viruses. Suitable microorganisms refer to any of those well established and those novel microorganisms and variants that emerge from time to time.

The term “spinning disk”, as used herein, or rotary atomizer, comprises a cylindrical disk that spins at high rpm and contains channels that discretize an incoming liquid feed into a number of thin streams that emanate radially from the disk as it spins. The discretized streams undergo Rayleigh breakup as they exit the disk. The spinning disk used in the examples was a Niro Mobile Minor air-driven rotary atomizer with a 2-inch disk.

A “system” refers to apparatus, components (such as biological actives or solutions) in the apparatus, and, optionally, conditions.

### BRIEF DESCRIPTION OF THE DRAWINGS

The objects, features and advantages of the present invention will be more readily appreciated upon reference to the following disclosure when considered in conjunction with the accompanying drawings.

FIG. 1: Spinning disk set-up for spraying alginate solution and biological actives. The spinning disk equipment was set-up to spray alginate solution and biological actives into a curing bath in the pan.

FIG. 2: Viability test of the microorganism. The viability of B. japonicum was evaluated under different test conditions described in Example 1, using the colony forming assay and quantified as colony forming units (CFU) as shown.

FIG. 3A is an image of B. japonicum prior to passage through the spinning disk equipment.

FIG. 3B is a representative image of B. japonicum after passage through the spinning disk equipment with a serrated cup.

FIG. 3C is a representative image of B. japonicum after passage through the spinning disk equipment with a smooth cup.

FIG. 3D is a representative image of B. japonicum after passage through the spinning disk equipment with a serrated cup with a cup speed of 30,000 rpm.

FIG. 4A is a representative image of of B. japonicum cells mixed with alginate solution and sprayed into 200 mM CaCl2 bath, before image acquisition as described in Test 6.

FIG. 4B is a representative image of of B. japonicum cells mixed with alginate solution and sprayed into 200 mM CaCl2 bath, before image acquisition. Cup speed was set to 30,000rpm as described in Test 7. Scale bar is 10 μm.

### DETAILED DESCRIPTION OF THE INVENTION

This disclosure describes a method and system to scale-up encapsulation of microorganisms and/or proteins, typically for use in promoting plant growth or seed protection.

In a method of this invention, a fermenter is used to culture microorganisms through the various phases of their physiological cycle. A fermenter is utilized for the cultivation of microbial cells, which may be maintained at particular phases in their growth curve. The use of fermenters is advantageous in many ways for cultivating optimal growth. Generally, the control of growth conditions including control of dissolved carbon dioxide, oxygen and other gases, dissolved nutrients, trace elements, temperature and pH is facilitated in a fermenter. To give an illustration of the application of a fermenter in certain embodiments of the present invention, a fermenter containing nutrient medium is inoculated with the microorganism. Generally, there will follow a lag phase prior to cells beginning to double. After the lag phase, the cell doubling time decreases and the culture goes into the logarithmic phase. The logarithmic phase is eventually followed by an increase of the doubling time that, while not intending to be limited by theory, is thought to result from either a mass transfer limitation, depletion of nutrients including nitrogen or mineral sources, or a rise in the concentration of inhibitory chemicals, or quorum sensing by the microbes. The growth slows down and ceases when the culture goes into the stationary phase. In order to harvest cell mass, the culture in certain embodiments is harvested in the logarithmic phase and/or the arithmetic phase and/or in the stationary phase.

Inoculation can be filled with a starting batch of nutrient media and/or additives at the beginning of growth and no additional nutrient media and/or additives are added after inoculation.

Inoculation of the culture into the fermenter can be performed by methods including, but not limited to, transfer of culture from an existing culture inhabiting another bioreactor, or incubation from a seed stock raised in an incubator. The seed stock of the strain may be transported and stored in forms including, but not limited to, a powder, liquid, frozen, or freeze-dried form as well as any other suitable form, which may be readily recognized by one skilled in the art. The reserve bacterial cultures can be kept in a metabolically inactive, freeze-dried state until required for restart. In certain embodiments when establishing a culture in a very large reactor, cultures can be grown and established in progressively larger intermediate scale vessels prior to inoculation of the full-scale vessel.

The fermenters have mechanisms to enable mixing of the nutrient media that include, but are not limited to, one or more of the following: spinning stir bars, blades, impellers, or turbines: spinning, rocking, or turning vessels; gas lifts, sparging; recirculation of broth from the bottom of the container to the top via a recirculation conduit, flowing the broth through a loop and/or static mixers. The culture media may be mixed continuously or intermittently.

Recipes to grow the microbe vary greatly in the same wide variety as microbes, as is well known in the art. For example, every species of bacteria has optimal growing conditions with a wide range of possible chemical compositions, and differences in the media used to grow them. A recipe will include at least any aspect that may possibly affect the growth of a microbe during its life cycle. A recipe may also include exemplary aspects such as, for example, additional additives, correlations if needed with possibly different stages of growth, or recommendations for scaling based on densities of microbes, optimal lighting, aerobic or anaerobic conditions and other environment factors including whether specific biosafety levels may be recommended.

One preferred microorganism is Bradyrhizobium japonicum. The method of this invention may be practiced with the Bradyrhizobium japonicum strain disclosed herein or, for example, with microorganisms Bradyrhizobium japonicum, Pseudomonas protegens, Rhizobium tropici, Rhizobium leguminosarum, Ensifer meliloti, Burkholderia phytofirmans, Azospirillum brasilense, Rhodopseudomonas acidophila, Rhodotorula sp., Penicillium bilaiae, or combinations thereof.

In methods of this invention, microbial cells can be mixed with sodium alginate solution. The cell/alginate solution is sprayed using a spinning disc sprayer into a crosslinking bath or curing bath, with or without bath agitation. The cell/alginate solution sprayed into the bath cures into microbeads.

In the context of the present invention, microbeads are understood to be microparticles which have a capsule shell or wall and at least one or more active ingredients as core material inside the microbead.

The spinning disc sprayer achieves flowrates up to ˜500 ml/min. Increasing rotation speeds of the spinning disk, achieves a proportionately smaller droplet size of the cell/alginate solution. Additionally, the serrated cup in the spinning disk divides the liquid into channels and as a result achieves smaller droplet sizes compared to use of a smooth cup, when all parameters in the experiment set-up are kept constant.

In some non-limiting embodiments, alginate microbead encapsulating microorganisms, exhibit particle sizes of 10 to 200 μm, or 20 to 100 μm, or 50 to 500 μm. Particle sizes are determined using laser diffraction such as by a Malvern Spraytec.

Combinations of the invention may also provide for an extended spectrum of activity in comparison to that obtained by each individual biological active component in the microbead, and/or permit the use of lower amounts of the individual components when used in combination to that when used alone, in order to mediate effective activity.

### Example 1: Large-Scale Production of Alginate Microbeads with Retained Viability

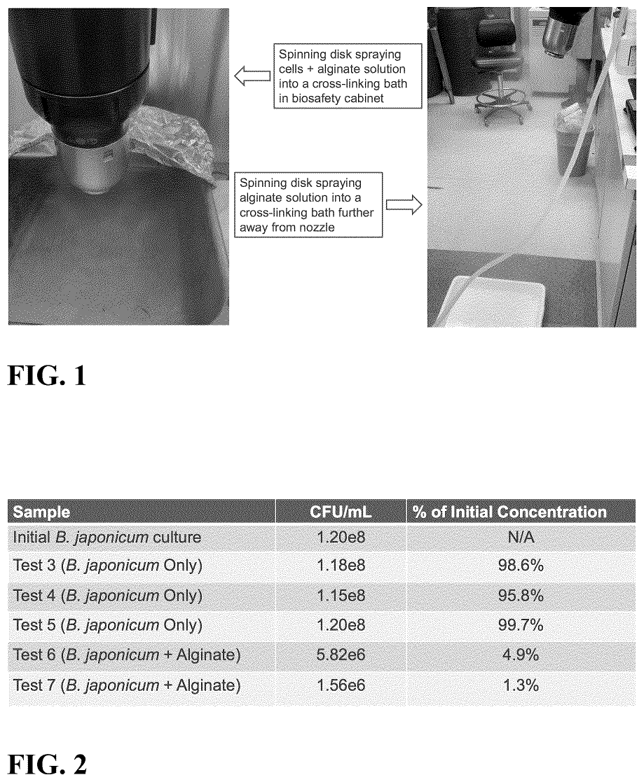

As shown in FIG. 1, using a spinning disk, the alginate solution (10 mg/ml or 40 mg/ml) alone or mixed with the microorganisms, or microorganisms only, was sprayed into the curing bath (200 mM CaCl₂). The following 8 tests were performed for the large-scale production of alginate microbeads.

Test 1: Alginate solution sprayed into 200 mM CaCl₂ bath 100 mL/min, 20 psi, 60,000 rpm, 50 mm serrated cup, circular glass dish for bath, 1 l stirred bath, sprayed 49 ml in the biosafety cabinet.

Test 2: Alginate solution sprayed into 200 mM CaCl₂ bath. Same parameters as Test 1 except airflow set to 30 psi.

Test 3: B. japonicum cells only (no alginate) sprayed with no bath. Same parameters as Test 1 except that 100 ml volume was sprayed into empty metal pan.

Test 4: B. japonicum cells only (no alginate) sprayed with no curing bath. Same parameters as Test 3 except used smooth cup instead of serrated.

Test 5: B. japonicum cells only (no alginate) sprayed with no bath. Same parameters as Test 3 except cup speed was at 30,000 rpm

Test 6: B. japonicum cells mixed with alginate solution sprayed into 200 mM CaCl₂ bath. Same parameters as Test 3 except that cross-linking solution was present in metal pan.

Test 7: B. japonicum cells mixed with alginate solution sprayed into 200 mM CaCl₂ bath. Same parameters as Test 6 except cup speed at 30,000 rpm.

Test 8: Alginate solution sprayed into 200 mM CaCl2 bath. Same parameters as Test 2 except sprayed outside of biosafety cabinet further away from bath.

### CFU (Colony Forming Unit) Measurement

Cultures were diluted serially in PBS buffer to obtain desired amount of Colony Forming Units (CFU) per millimeter. The bacteria were quantified by plating appropriate dilutions on agar plates and incubated overnight at 30° C. The colonies grown on plates were counted. Final CFU/ml was calculated by multiplying the colony count with the dilution factor.

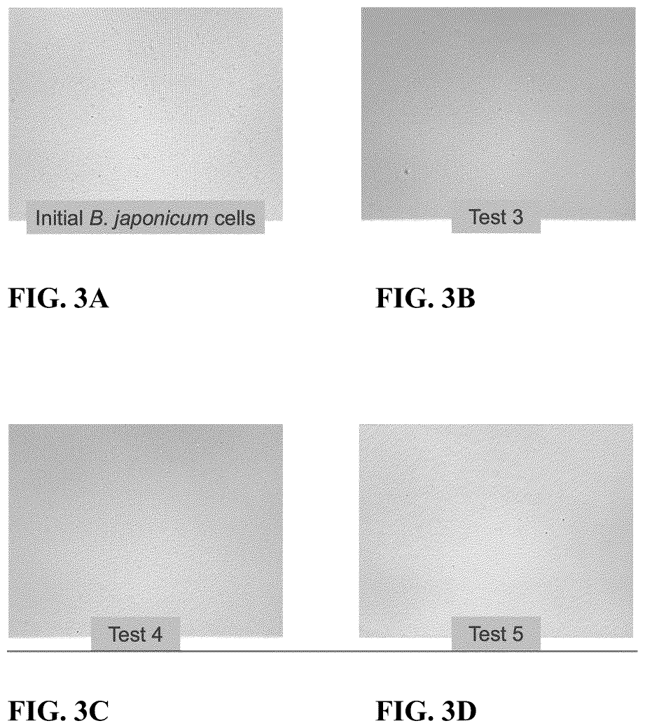

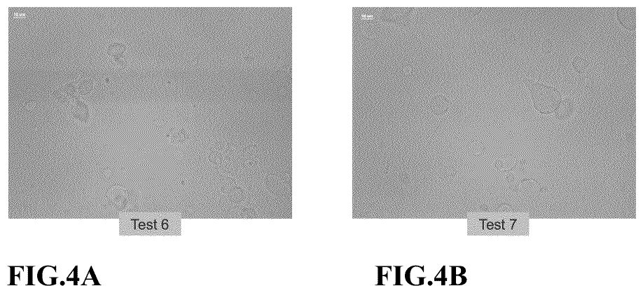

As shown in FIG. 2, the viability of the microorganism remained unaffected by the spinning disk equipment and retained >95% cell viability. The morphology of the microorganisms was also retained by this method and resembled its original morphology prior to spraying by the spinning disk (compare FIG. 3A to FIG. 3B-3D). Tests 6 and 7 that generated alginate microbeads encapsulating B. japonicum produced several microbeads in the size range under 100 μm (FIG. 4A-4B).

What is claimed is:

1. A system or method for generation of alginate microbeads encapsulating biological actives, comprising passing a solution comprising a biological active and an amphiphilic compound onto a spinning disk to generate microbeads and capturing the microbeads in a curing bath.

2. The method of claim 1 wherein the “biological active” comprises a protein.

3. The method of claim 1 wherein the biological active is selected from the group comprising: Bradyrhizobium japonicum, Pseudomonas protegens, Rhizobium tropici, Rhizobium leguminosarum, Ensifer meliloti, Burkholderia phytofirmans, Azospirillum brasilense, Rhodopseudomonas acidophila, Rhodotorula sp., Penicillium bilaiae, and combinations thereof.

4. The method of claim 1 wherein the amphiphilic compound is a polysaccharide.

5. The method of claim 4 wherein the amphiphilic compound is selected from the group consisting of sodium alginate, potassium alginate, barium alginate, calcium alginate, magnesium alginate, strontium alginate, and a combination thereof.

6. The method of claim 1 wherein the curing bath comprises an aqueous solution comprising calcium chloride.

7. The method of claim 1 wherein the curing bath comprises an aqueous solution comprising calcium acetate.

8. The method of claim 1 wherein at least 95 mass % of the microbeads are in the size range of 1 to 800 μm, or 1 to 500 μm.

9. The method of claim 8 wherein at least 30 mass %, or at least 50 mass %, at least 70 mass %, or at least 90 mass % of the microbeads are in the size range of 50 to 100 μm.

10. The method of claim 1 wherein at least 80 mass %, or at least 90 mass % of the microbeads are spherical.

11. The method of claim 1 wherein the spinning disk comprises a serrated cup.

12. The method of claim 11 wherein the spinning disk retains the viability of the microorganisms.

13. The method of claim 12 wherein at least 30 mass %, or at least 50 mass %, at least 70 mass %, or at least 90 mass % of the microbeads exhibit at least 95% viability.

14. The method of claim 1 comprising generating 1000 liters of encapsulated biological actives while retaining at least 80% of activity.
